(19) United States
(12) Patent Application Publication
(10) Pub. No.: US 2024/0148752 A1
(43) Pub. Date: May 9, 2024

(54) Canker sore preparation for the treatment of canker sore.

(71) Applicant: SAIF UL ISLAM, Sacramento, CA (US)

(72) Inventor: SAIF UL ISLAM, Sacramento, CA (US)

(21) Appl. No.: 17/981,306

(22) Filed: Nov. 4, 2022

(51) Int. Cl. A61K 31/573; A61K 9/00; A61K 31/245

(52) U.S. Cl. CPC A61K 31/573; A61K 9/006; A61K 31/245

(57) ABSTRACT

Present invention discloses a topical canker sore preparation, containing combination of hydrocortisone and benzocaine as a novel, and effective active ingredients for the treatment of canker sore.

Present invention is related to the field of inventions that are used for the treatment of canker sore.

### BACKGROUND

There are several products available that are used for canker sore. Most of the products contained either benzocaine or lidocaine. One prescription product has triamcinolone in orabase. Some other products have herbal or homeopathic ingredients in it. None of the oral product is the combination of hydrocortisone and benzocaine or lidocaine. This invention is the first product that is used for canker sore and have both anti-inflammatory properties and pain reliever.

### Pathophysiology of Canker Sore:

Exact cause of canker sore is not known but it is proposed to be multifactor. Microscopic observation of the canker sore show dense accumulation of T-cells which resulted in inflammatory response involving generation of interleukins, TNF etc. Several other studies have shown strong evidences of T-cell mediated immunological response which resulted in ulcer and pain in the area

Hydrocortisone has anti-inflammatory properties which stop the progression of the inflammatory response and fasten the recovery. Benzocaine or lidocaine control the pain.

Advantage: Present invention will not only relief the pain but also hasten the healing process. None of the available oral products in the market have both characteristics.

### DETAIL DESCRIPTION

This invention relates to over-the-counter medication to treat canker sore. Canker sore which is also called aphthous stomatitis is a painful ulceration which usually recur several time. The problem is not infectious but the over acting immune cells, particularly T cells, and other inflammatory molecules. Hydrocortisone or other corticosteroid are strong anti-inflammatory agents and inhibits these inflammatory molecules. Therefore, fasten the recovery. Triamcinolone in orabase which is a prescription medicine, use the same concept and is used for canker sore to decrease the inflammation. However, this product does not have any pain reliever in it. Patient still fell pain through the duration of canker sore. Then we have products that have local anesthetics such as benzocaine or lidocaine. These products relief the pain but do nothing with the inflammation or healing.

Present invention has combined both anti-inflammatory agent and a pain reliever into one. Therefore, have a property of relieving the pain and decrease the inflammation, which result in better treatment of canker sore.

Invention contain 1% hydrocortisone one part by weight and one part by weight of 20% benzocaine. However, hydrocortisone can be substitute to any other corticosteroid agents, and 20% benzocaine can be substituted with other strength or lidocaine.

Both ingredients 1% hydrocortisone and 20% benzocaine are mixed in 1 to 1 ratio or to any other suitable ratio. Inactive ingredients used are, orabase, allantoin, carbomer, disodium EDTA, polyethylene glycol, color, are added to make the product better absorb and appealing.

### SUMMARY OF THE INVENTION

In view of this, curative effect of the invention is obvious in view of the deficiencies of prior art. It is the objective of present invention to provide canker sore treatment that have anti-inflammatory and analgesic properties to fasten healing process and relief the pain.

In order to achieve above objectives, the invention adopts follow technical scheme.
Canker sore preparation, ointment, cream, lotion, or gel include following ingredients parts by weight.
   1 part by weight of 1% hydrocortisone
   1 part by weight of 20% benzocaine.
   1% hydrocortisone is mixed homogeneously with 20% benzocaine.
   Inactive ingredients such as allantoin, carbomer, disodium EDTA, flavor, polyethylene glycol, polysorbate 60, propylene glycol, sodium saccharin, sorbic acid, stearyl alcohol, and water are mixed all together.
   Final product could be an ointment or cream or lotion or gel.
   Apply topically three times or four times daily at the canker sore in a mouth, gum, soft palate, tongue, or any other affected site.
   The beneficial effects of the invention are:
      Anti-inflammatory properties, which can reduce the inflammation at the affected site and result in faster healing.
      Analgesic property

### Embodiments of the Invention

Technical scheme of present invention is further described in the following embodiments, but the present invention simultaneously is not limited to these embodiments.

### Embodiment 1

Present invention may include other agents such as zinc, cinnamon, or any other herbals or chemicals.

### Embodiment 2

Present invention may include any other herbal or chemicals help for pain.

### Embodiment 3

Present invention can be an ointment or cream or lotion or gel or any other possible form that can be applied on the affected area.

### Embodiment 4

Present invention can be mixed in different ways, such as 1 part by weight of hydrocortisone and 2 part by weight of benzocaine or 2 part by weight of hydrocortisone and 1 part by weight of benzocaine.

### Embodiment 5

Present invention can have a cooling agent(s) mixed, such as menthol.
Finally, it is stated that the above examples are only used to illustrate the technical scheme of present invention and are not limiting.

1. Canker sore preparation containing the combination of active ingredients hydrocortisone, and benzocaine to treat canker sore in oral cavity, gums, mouth, lips, or any other mucus membrane.

2. Canker sore preparation of claim 1 contain 1%-10% or higher concentration of hydrocortisone.

3. Canker sore preparation of claim 1 contain 1 part by weight of hydrocortisone.

4. Canker sore preparation of claim 1 contain 2 part by weight of hydrocortisone

5. Canker sore preparation of claim 1 contain 20%-40% benzocaine.

6. Canker sore preparation of claim 1 contain 1 part by weight benzocaine

7. Canker sore preparation of claim 1 contain 2 part by weight benzocaine

8. Canker sore preparation of claim 1 hydrocortisone can be replaced by any other corticosteroid, such as but not limited to triamcinolone, fluocinolone, desonide, betamethasone, alclometasone, fluticasone, flurandrenolide, mometasone, clocortolone, fluocinonide, diflucortolone, dexamethasone, diflorasone, amcinonide, halobetasol, halcinonide, clobetasol

9. Canker sore preparation of claim 1, benzocaine can be replaced by lidocaine.

10. Canker sore preparation of claim 1 is applied topically at the site of canker sore.

11. Canker sore preparation of claim 10 is applied in the form of ointment

12. Canker sore preparation of claim 10 is applied in the form of cream

13. Canker sore preparation of claim 10 is applied in the form of gel

14. Canker sore preparation of claim 10 is applied in the form of lotion

15. Canker sore preparation of claim 10 is applied in the form of foam
